(19) United States
(12) Patent Application Publication
(10) Pub. No.: US 2019/0108451 A1
(43) Pub. Date: Apr. 11, 2019

(54) COGNITIVE HEALTH CARE VITAL SIGN DETERMINATION TO NEGATE WHITE COAT HYPERTENSION IMPACT

(71) Applicant: INTERNATIONAL BUSINESS MACHINES CORPORATION, Armonk, NY (US)

(72) Inventors: Kyle R. MOSER, Stone Ridge, NY (US); Mark D. SMITH, Austin, TX (US); Andrew P. WACK, Millbrook, NY (US); Maria R. WARD, Pflugerville, TX (US)

(21) Appl. No.: 15/800,196

(22) Filed: Nov. 1, 2017

Related U.S. Application Data
(63) Continuation of application No. 15/729,290, filed on Oct. 10, 2017.

(51) Int. Cl. G06N 5/04; A61B 5/021; A61B 5/00

(52) U.S. Cl. CPC G06N 5/04; G06N 99/005; A61B 5/4076; A61B 5/021

(57) ABSTRACT

Embodiments include methods, systems, and computer program products for determining health care vital data. Aspects include receiving a health care vital measurement for a patient and health care vital data for a population. Aspects also include determining a baseline for the patient based at least in part upon the health care vital data for the population. Aspects include determining whether the patient health care vital data deviates from the baseline by more than a threshold and, applying a cognitive learning model to the patient health care vital measurement to correct for an anxiety-based impact to generate a corrected health care vital measurement responsive to a determination that the patient health care vital data deviates from the baseline by more than the threshold.

### DOMESTIC AND/OR FOREIGN PRIORITY

This application is a continuation of U.S. application Ser. No. 15/729,290, titled “Cognitive Health Care Vital Sign Determination to Negate White Coat Hypertension Impact” filed Oct. 10, 2017, the contents of which are incorporated by reference herein in its entirety.

### BACKGROUND

The present invention relates to cognitive health care vital signs, and more specifically, to cognitive health care vital sign determination to negate anxiety impact.

Independent of actual or perceived health status and demographic characteristics, when visiting a health care professional, many individuals experience increased levels of anxiety associated with the visit itself. This anxiety has been referred to as “white coat syndrome” and includes increased levels of anxiety when visiting and interacting with health care professionals. Such increased levels of anxiety can result in elevated or erroneous health care vital sign determinations, which can lead, for instance, to misdiagnosis of medical conditions. The results and impact of erroneous or inaccurate health care vital sign determinations can range from modest to severe.

### SUMMARY

In accordance with one or more embodiments, a computer-implemented method for determining health care vital data is provided. The method includes receiving, by a processor, a health care vital measurement for a patient. The method also includes receiving, by the processor, health care vital data for a population. The method also includes determining, by the processor, a baseline for the patient based at least in part upon the health care vital data for the population. The method also includes determining, whether the patient health care vital data deviates from the baseline by more than a threshold. The method also includes responsive to a determination that the patient health care vital data deviates from the baseline by more than the threshold, applying a cognitive learning model to the patient health care vital measurement to correct for an anxiety-based impact to generate a corrected health care vital measurement. The method also includes outputting the corrected health care vital measurement.

In accordance with another embodiment, a computer program product for determining health care vital data is provided. The computer program product includes a computer readable storage medium readable by a processing circuit and storing program instructions for execution by the processing circuit for performing a method. The method includes receiving a health care vital measurement for a patient. The method also includes receiving health care vital data for a population. The method also includes determining a baseline for the patient based at least in part upon the health care vital data for the population. The method also includes determining whether the patient health care vital data deviates from the baseline by more than a threshold. The method also includes, responsive to a determination that the patient health care vital data deviates from the baseline by more than the threshold, applying a cognitive learning model to the patient health care vital measurement to correct for an anxiety-based impact to generate a corrected health care vital measurement. The method also includes outputting the corrected health care vital measurement.

In accordance with a further embodiment, a processing system for determining health care vital data is provided. The processing system includes a processor in communication with one or more types of memory. The processor is configured to receive a health care vital measurement for a patient. The processor is also configured to receive health care vital data for a population. The processor is also configured to determine a baseline for the patient based at least in part upon the health care vital data for the population. The processor is also configured to determine whether the patient health care vital data deviates from the baseline by more than a threshold. The processor is also configured to, responsive to a determination that the patient health care vital data deviates from the baseline by more than the threshold, apply a cognitive learning model to the patient health care vital measurement to correct for an anxiety-based impact to generate a corrected health care vital measurement. The processor is also configured to output the corrected health care vital measurement.

### BRIEF DESCRIPTION OF THE DRAWINGS

The subject matter of the present invention is particularly pointed out and distinctly claimed in the claims at the conclusion of the specification. The foregoing and other features and advantages of the one or more embodiments described herein are apparent from the following detailed description taken in conjunction with the accompanying drawings in which:

FIG. 1 depicts a cloud computing environment according to an embodiment of the present invention.

FIG. 2 depicts abstraction model layers according to an embodiment of the present invention.

FIG. 3 depicts a computer system according to one or more embodiments of the present invention.

FIG. 4 is a flow diagram illustrating a method for health care vital sign determination according to one or more embodiments of the present invention.

FIG. 5 depicts a diagram illustrating an exemplary system for health care vital sign determination according to one or more embodiments of the present invention.

### DETAILED DESCRIPTION

It is understood in advance that although this description includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model can include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but can be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It can be managed by the organization or a third party and can exist on-premises or off-premises.

Community cloud: the cloud infrastructure is shared by several organizations and supports a specific community that has shared concerns (e.g., mission, security requirements, policy, and compliance considerations). It can be managed by the organizations or a third party and can exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure including a network of interconnected nodes.

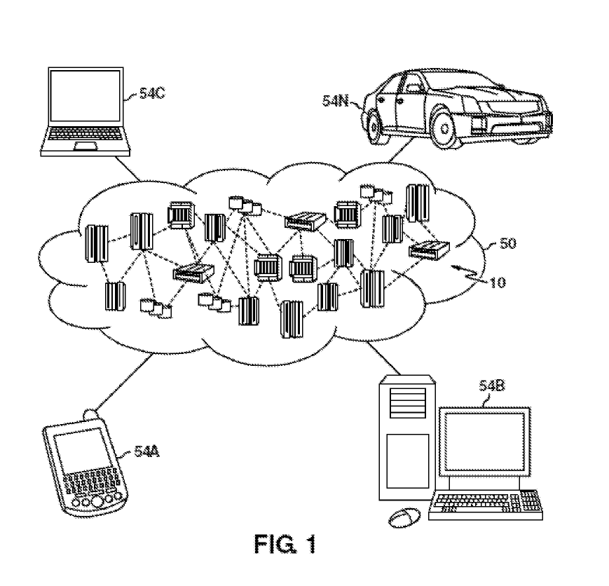

Referring now to FIG. 1, illustrative cloud computing environment 50 according to one or more embodiments of the present invention is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N can communicate. Nodes 10 can communicate with one another. They can be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 1 are intended to be illustrative only and that computing nodes 10 and cloud computing environment 50 can communicate with any type of computerized device over any type of network and/or network addressable connection (e.g., using a web browser).

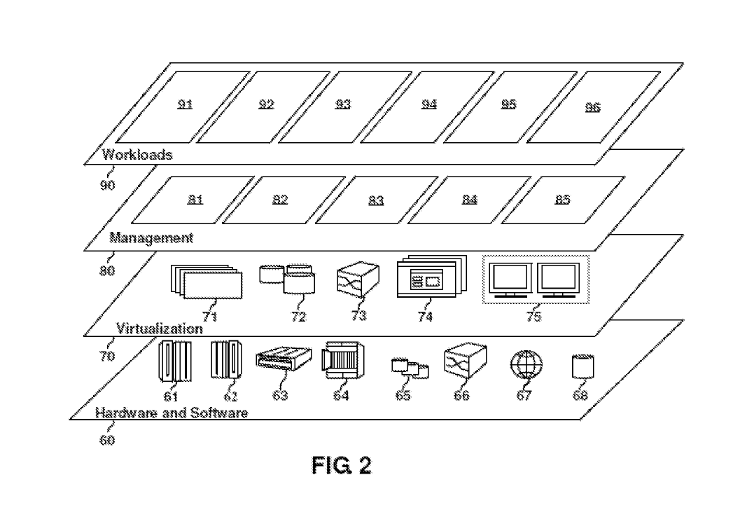

Referring now to FIG. 2, a set of functional abstraction layers provided by cloud computing environment 50 (FIG. 1) according to one or more embodiments of the present invention is shown. It should be understood in advance that the components, layers, and functions shown in FIG. 2 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities can be provided: virtual servers 71; virtual storage 72; virtual networks 73, including virtual private networks; virtual applications and operating systems 74; and virtual clients 75.

In one example, management layer 80 can provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources can include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment can be utilized. Examples of workloads and functions which can be provided from this layer include: mapping and navigation 91; software development and lifecycle management 92; virtual classroom education delivery 93; data analytics processing 94; transaction processing 95; and health care vital sign measurement analysis 96.

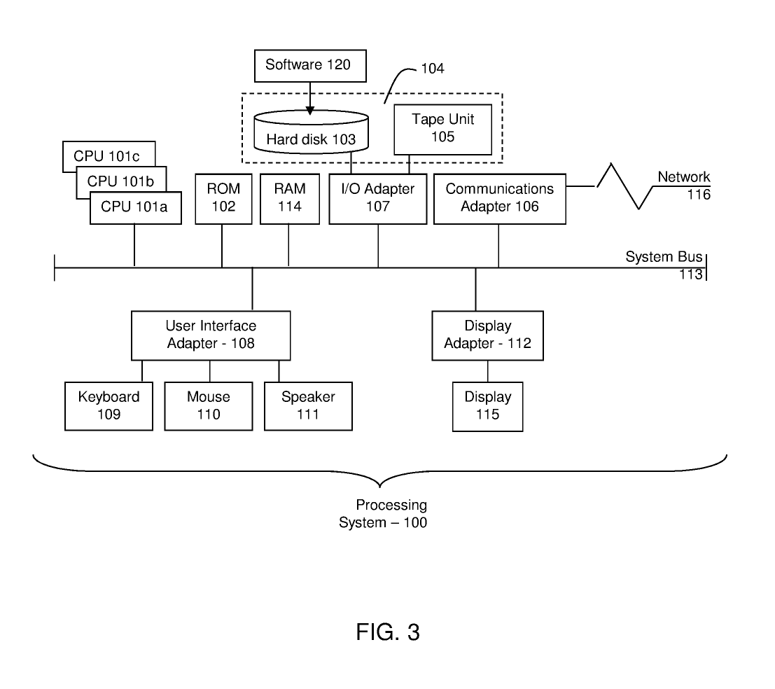

Referring now to FIG. 3, a schematic of a cloud computing node 100 included in a distributed cloud environment or cloud service network is shown according to one or more embodiments of the present invention. The cloud computing node 100 is only one example of a suitable cloud computing node and is not intended to suggest any limitation as to the scope of use or functionality of embodiments of the invention described herein. Regardless, cloud computing node 100 is capable of being implemented and/or performing any of the functionality set forth hereinabove.

In cloud computing node 100 there is a computer system/server 12, which is operational with numerous other general purpose or special purpose computing system environments or configurations. Examples of well-known computing systems, environments, and/or configurations that can be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 can be described in the general context of computer system-executable instructions, such as program modules, being executed by a computer system. Generally, program modules can include routines, programs, objects, components, logic, data structures, and so on that perform particular tasks or implement particular abstract data types. Computer system/server 12 can be practiced in distributed cloud computing environments where tasks are performed by remote processing devices that are linked through a communications network. In a distributed cloud computing environment, program modules can be located in both local and remote computer system storage media including memory storage devices.

As shown in FIG. 3, computer system/server 12 in cloud computing node 100 is shown in the form of a general-purpose computing device. The components of computer system/server 12 can include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media can be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 can further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 can include at least one program product having a set (e.g., at least one) of program modules that are configured to carry out the functions of embodiments of the invention.

Program/utility 40, having a set (at least one) of program modules 42, can be stored in memory 28 by way of example, and not limitation, as well as an operating system, one or more application programs, other program modules, and program data. Each of the operating system, one or more application programs, other program modules, and program data or some combination thereof, can include an implementation of a networking environment. Program modules 42 generally carry out one or more functions and/or methodologies in accordance with some embodiments of the present invention.

Computer system/server 12 can also communicate with one or more external devices 14 such as a keyboard, a pointing device, a display 24, etc., one or more devices that enable a user to interact with computer system/server 12, and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

Embodiments of the invention relate to determining more accurate health care vital signs to negate white coat hypertension impact. When visiting a health care professional, such as a doctor, nurse, or physician's assistant, for a regular or non-regular checkup, many individuals experience what has been commonly referred to as “white coat syndrome,” which includes anxiety associated with the visit to and/or the interaction with health care professionals. This anxiety can be independent of age, race, ethnicity, weight, actual and/or perceived health status. The increased levels of anxiety associated with white coat syndrome can result in elevated or abnormal health care vital sign measurements, such as blood pressure, heart rate, respiration rate, and the like.

White coat hypertension can, thus, result in misdiagnosis of medical conditions, with potentially serious consequences. For example, an elevated blood pressure or other vital sign measurements resulting from white coat syndrome can lead to prescribing incorrect medications or medication dosages. Incorrect prescriptions can cause, for example, inconvenience to patients and caregivers in acquisition of medications, unnecessary and/or excessive financial expenditures on the part of patients, insurance providers, governments, and care givers, and can have potentially significant medical impacts on patients, including adverse health effects. Moreover, incorrect vital sign measurements can lead to an incorrect analysis of local, regional, national and global healthcare trends and can potentially mask serious underlying health issues.

Embodiments of the present invention provide systems and methods to accurately assess basic health care vital signs in the course of direct interaction with health care professionals, where many patients experience some level of anxiety impacting their vital signs independent of health status.

Embodiments of the invention can use cognitive methodologies based upon machine learning of a variety of general population-based and patient-specific factors and assessments coupled with baseline vitals for an individual patient can be used to more accurately determine a patient's actual or corrected vital signs during a health care visit. For example, individual or population-based age, gender, race, weight, geography, and general and specific health history, including medications, anxiety assessments, home vital sign assessments and similar information can be collected and coupled with baseline vitals for an individual patient. Using such information, cognitive technologies, such as Watson, can assess whether and to what degree elevated or abnormal vital sign measurements are caused by real health issues as opposed to anxiety associated with white coat hypertension.

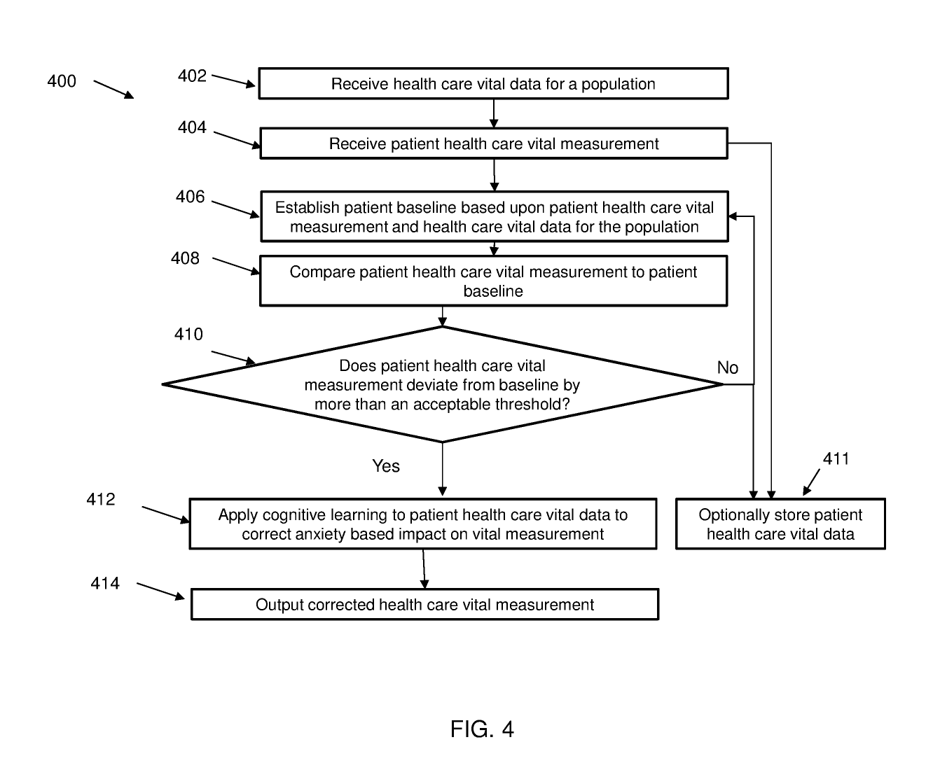

Referring now to FIG. 4, a flow chart illustrating an exemplary method 400 for determining health care vital data according to one or more embodiments of the present invention is shown. The method 400 includes, as shown at block 402 receiving health care vital data for a population. The method also includes, as shown at block 404, receiving health care vital data. As shown at block 406, the method 400 also includes establishing a patient baseline based upon patient health care vital data and health care vital data for the population. In some embodiments of the invention, establishing a patient baseline includes obtaining several vital sign measurements over an appropriate time period or over appropriate intervals. In some embodiments of the invention, the patient baseline is based at least in part upon a plurality of historic health care vital measurements for the patient. The historic health care vital measurements for the patient can be obtained within the same day or over a longer period of time, such as over weeks, months, or years.

The method 400 also includes, as shown at block 408, comparing patient health care vital data to the patient baseline. As shown at decision block 410, the method 400 asks whether the patient health care vital measurement deviates from the baseline by more than an acceptable threshold. The acceptable threshold can be, for example, a pre-determined threshold or a statistically determined threshold above which the vital measurement is likely to be indicative of a health issue. Responsive to a determination that the measurement deviates from the baseline by more than an acceptable threshold, the method 400 can proceed to block 412 and applies cognitive learning to the patient health care vital data to correct an anxiety-based impact on the vital measurement. For example, the method can apply an automated mechanism for searching through large sets of sources of content, such as with the Watson system available from International Business Machines (IBM) Corporation and other machine learning technologies. Such sources of content include health care population data and available patient health care vital data and measurements, to determine and/or quantify any anxiety-based impact on the vital sign measurement, such as elevated blood pressure due to white coat syndrome, and provide a corrected vital sign measurement that removes any anxiety-based impact. In some embodiments of the invention, cognitive learning uses an extensive array of the individual patient's health information to determine any changes in health that are not attributed to stress and/or anxiety associated with the clinical visit.

The method 400 also includes outputting a corrected health care vital measurement, as shown at block 414. The method 400 can also include optionally storing patient health care vital data, as shown at block 411, for instance to a secured database that is consistent with applicable legal protections and privacy requirements (e.g., Health Insurance Portability and Accountability Act of 1996 (HIPPA)). For example, the method can include storing patient health care vital data upon receipt of a patient health care vital measurement, after establishing a patient baseline, and/or after a determination that the measurement does not deviate from the baseline by more than a threshold. In some embodiments of the invention, patient health care vital data can be stored with related comparison information, testing information, medical history information, population health care vital information, or any other information that could be useful in determining a corrected vital sign measurement.

Health care vital measurement include medical measurements that indicate the state of a patient's body functions and that could be affected by anxiety or stress. For example, health care vital data can include pulse rate or regularity, respiration rate, blood pressure, sugar levels, and blood oxygen levels. Other medical measurements that can be impacted by stress, and the manner in which anxiety or stress impacts each medical measurement, are known to persons skilled in the art. For instance, it is known that anxiety can elevate pulse rate, respiration rate, and blood pressure.

Health care vital data can include health care vital measurements and related data, such as testing conditions and location (for instance clinic versus home testing), patient medical data and medical history, patient demographic data (such as age, gender, ethnicity, weight, geographic locations, etc.).

The population from which the health care vital data is obtained can be tailored to the individual patient to provide a relevant comparison. For example, the population can include a subset of individuals within a relevant geographic area, such as on a local, regional, national, or international level. The population can include a subset of individuals with the same or similar demographic information and/or medical background. The population can include, in some embodiments of the invention, individuals with white coat anxiety levels known to be comparable to the patient.

In some embodiments of the invention, a method includes obtaining, for example responsive to a determination that the patient health care vital data deviates from the baseline by more than the threshold, a controlled health care vital measurement for the patient. For example, embodiments of the invention can notify the clinician or health care provider of a potential anxiety-based impact to vital sign measurements. The health care provider can then instruct the patient to obtain relevant vital sign measurements in a controlled setting and/or non-stressful environment, such as by self-measurement or by measurement via an automated machine at a pharmacy or other non-clinical setting. In some embodiments of the invention, after obtaining a controlled or non-stressful environment measurement, a method can include updating the baseline for the individual patient. In some embodiments of the invention, after obtaining a controlled health care vital measurement for the patient, the method proceeds to compare the patient's vital sign measurement to an updated baseline.

In some embodiments of the invention, necessary medications and health care regimens are provided responsive to the corrected vital sign measurements. Such medications and regimens can be more appropriate to the individual patient than those that would have been prescribed absent a corrected vital sign measurement, for example, in the case where an elevated blood pressure is an artifact of white coat syndrome, embodiments of the invention can identify such anxiety-based artifacts and prevent needless prescription of blood pressure related medicines.

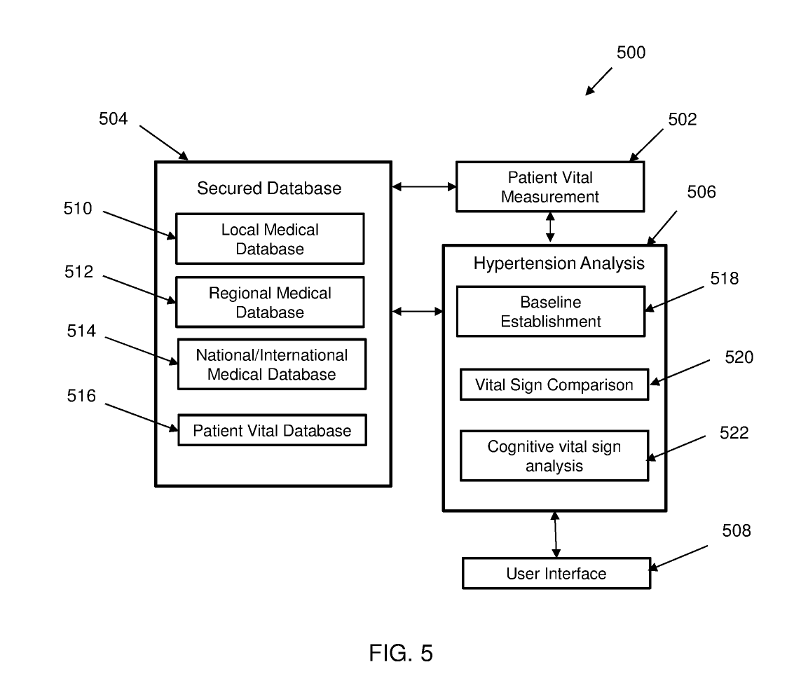

FIG. 5 illustrates an exemplary system 500 for determining health care vital signs according to one or more embodiments of the present invention. The system can include, for example, a secured database 504, a hypertension analysis engine 506, and a user interface 508 in communication with the hypertension analysis engine 506. The secured database 504 can include a local medical database 510, a regional medical database 512, a national and/or international medical database 514, and a patient vital database 516. The aforementioned databases can include health are vital sign data for populations or subsets of populations, as appropriate. A patient vital measurement 502 can be accessed by the secured database 504 and/or the hypertension analysis engine 506. The hypertension analysis engine 506 can include a baseline establishment module 518, for establishing a baseline. The hypertension analysis engine 506 can also include a vital sign comparison module 520 and a cognitive vital sign analysis module 522. The user interface 508 can include any graphical, textual, auditory feedback system, such as a graphical user interface.

For example, if a patient has an elevated blood pressure and heart rate and has gained a moderate to significant amount of weight, embodiments of the invention can provide a list of reasons for the elevated blood pressure and heart rate along with a distribution (for instance a percent distribution) for the elevated vitals. In this example, weight gain alone can lead to both elevated blood pressure and elevated heart rate. Embodiments of the invention can assess whether the weight gain alone correlates with and resulted in the elevated blood pressure and heart rate or, for example, whether the elevated vitals can also be attributed to anxiety. Such assessments can improve medication dosing for the patient, for example.

Conversely, in another example, for a patient having elevated blood pressure and heart rate with no change in weight, lifestyle, or other major causative factors, embodiments of the invention can provide reasonable insight into likely causes, including projections for more rare issues and/or identification of anxiety associated with the health care provider visit.

Embodiments of the invention can assess whether elevated vital signs exceed those associated with anxiety, thereby revealing potential medical issues that might otherwise be attributed to existing anxiety. For example, a patient that is informed of a cancer diagnosis could be expected to experience anxiety and, in conjunction with the anxiety, could be expected to have elevated blood pressure. Embodiments of the invention can determine whether the elevation in blood pressure can be solely attributed to anxiety associated with the diagnosis. For instance, based upon comparison of the individual blood pressure with the patient baseline, embodiments of the invention can determine that the elevation in blood pressure exceeds the elevation that can reasonably be attributed to anxiety for an individual patient. According, a health care professional can investigate other potential causes for the elevated blood pressure that might otherwise have been masked by the existence of anxiety.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form described. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The flow diagrams depicted herein are just one example. There can be many variations to this diagram or the steps (or operations) described therein without departing from the spirit of embodiments of the invention. For instance, the steps can be performed in a differing order or steps can be added, deleted or modified. All of these variations are considered a part of the claimed invention.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method for determining health care vital data, the method comprising:
receiving, by a processor, a patient health care vital measurement for a patient;
receiving, by the processor, health care vital data for a population;
determining, by the processor, a baseline for the patient based at least in part upon the health care vital data for the population;
determining, by the processor, whether the patient health care vital data deviates from the baseline by more than a threshold;
applying a cognitive learning model to the patient health care vital measurement to correct for an anxiety-based impact to generate a corrected health care vital measurement responsive to a determination that the patient health care vital data deviates from the baseline by more than the threshold; and
outputting the corrected health care vital measurement.

2. The computer-implemented method of claim 1, further comprising, receiving, by the processor, a controlled health care vital measurement for the patient responsive to a determination that the patient health care vital data deviates from the baseline by more than the threshold.

3. The computer-implemented method of claim 2, further comprising generating, by the processor, an updated baseline for the patient based at least in part upon the controlled health care vital measurement for the patient.

4. The computer-implemented method of claim 1, wherein the patient baseline is based at least in part upon a plurality of historic health care vital measurements for the patient.

5. The computer-implemented method of claim 1, wherein the population comprises a plurality of individuals having a shared demographic group to the patient.

6. The computer-implemented method of claim 1, wherein the population comprises a plurality of individuals having a shared medical condition to the patient.

7. The computer-implemented method of claim 1, further comprising storing one or more of the health care vital measurement, the determination that patient health care vital data deviates from the baseline by more than a threshold, or a determination that patient health care vital data does not deviate from the baseline by more than a threshold to a database.
